(19) United States
(12) Patent Application Publication
(10) Pub. No.: US 2011/0099087 A1
(43) Pub. Date: Apr. 28, 2011

(54) HOSGATE

(76) Inventor: WILLIAM R. REINHARDT, JR., San Clemente, CA (US)

(21) Appl. No.: 12/910,713

(22) Filed: Oct. 22, 2010

Related U.S. Application Data
(60) Provisional application No. 61/254,158, filed on Oct. 22, 2009.

(51) Int. Cl. G06Q 30/00

(52) U.S. Cl. 705/26.81

(57) ABSTRACT

A system and method for ordering, delivering and billing of the medical equipment utilizing the electronic data flow management system is disclosed. The system comprises at least one healthcare provider having at least one workstation, a computer ordering server, an online database and at least one durable medical equipment (DME) company having a mainframe server. The workstation generates and sends an order for the medical equipment. The computer ordering server receives the order from the workstation. The online database receives and stores the order from the computer ordering server. The mainframe server receives the order from the online database and delivers the medical equipment to a patient. The DME company utilizes a billing module to generate a bill for the medical equipment. The electronic data flow management system provides a secure and cost effective way of procuring the medical equipment to the healthcare provider.

### BACKGROUND OF THE INVENTION

This application claims the benefit of U.S. Provisional Application No. 61/254,158 filed on Oct. 22, 2010.

### TECHNICAL FIELD OF THE INVENTION

The present invention relates in general to a system and method for procuring medical equipment in healthcare organizations. More specifically, the present invention relates to an improved system and method for procuring medical equipment in a cost effective and secure way in healthcare organizations.

### DESCRIPTION OF THE RELATED ART

The rapid technological advances in the healthcare industry have led to development of improved and highly efficient medical equipment. This medical equipment provides significant healthcare benefits to patients. However, most of the healthcare organizations are normally not able to efficiently deliver required healthcare because of the exorbitant costs of the medical equipment procurement process.

Currently, the medical equipment procurement process for most healthcare facilities is woefully inefficient. Healthcare professionals normally assume responsibility for procuring medical equipment for their specific departments/patients because most healthcare facilities do not have a centralized department responsible for procuring the required medical equipment.

Conventional methods for procuring medical equipment have considerable drawbacks. For example, these methods use printed and/or verbal. communications to transmit orders, authorizations, and reauthorizations for medical equipment from healthcare providers like hospitals, independent practice associations, doctors, nurses, nursing facilities, pharmacies, government agencies and/or municipalities, and other hospital affiliated organizations to vendors of the medical equipment. Another major drawback in the existing methods is that these methods are time consuming and require too much labor and administrative overview. Furthermore, the cost for the resources required to complete these procurement tasks eventually trickles down to the patients adding, additional unnecessary healthcare costs.

Therefore, there is a need for a method that would provide a cost effective and secure way of procuring medical equipment. Such a needed method would use an efficient electronic communication means to collect and organize the order for medical equipment. Moreover, such a method would be simple and would provide real-time delivery of the medical equipment to the healthcare facilities/patients.

### SUMMARY OF THE INVENTION

To minimize the limitations found in the prior art, and to minimize other limitations that will be apparent upon the reading of the specifications, the present invention provides an electronic data flow management system for streamlining a process of ordering, delivering and billing of at least one medical equipment. The electronic data flow management system comprises at least one healthcare provider having at least one workstation, a computer ordering server associated with the at least one healthcare provider, an online database and at least one durable medical equipment (DME) company having a mainframe server. The at least one workstation is adaptable to generate and send an order for the at least one medical equipment. The computer ordering server receives the order for the at least one medical equipment from the at least one workstation. The online database receives and stores the order from the computer ordering server. The mainframe server receives the order from the online database and delivers the at least one medical equipment to at least one patient. The at least one DME company utilizes a billing module to generate a bill for the at least one medical equipment. The electronic data flow management system provides a secure and cost effective way of procuring the medical equipment to the healthcare provider.

In another aspect of the present invention, a method in accordance with the present invention is a method for ordering, delivering and billing of the medical equipment utilizing the electronic data flow management system. The healthcare provider utilizes the workstation to generate the order for the medical equipment. The workstation sends the order to the computer ordering server. The computer ordering server receives the order and sends to the online database. The online database receives and stores the order and directs the order to the mainframe server. The DME company receives the order and delivers the medical equipment to the patient. After delivering the medical equipment, the DME company sends the bill of the medical equipment to the healthcare provider. The healthcare provider receives the bill and the DME company sends a reauthorization request to the healthcare provider. The healthcare provider receives and verifies the reauthorization request, approves the reauthorization request and thereafter sends the approval to the DME company. The DME company receives and stores the approval for the reauthorization request.

One objective of the invention is to provide an electronic data flow management system for streamlining a process of ordering, delivering and billing of at least one medical equipment.

Another objective of the invention is to provide a secure and cost effective way of procuring at least one medical equipment to at least one healthcare provider.

A third objective of the invention is to provide a method that uses an efficient electronic communication means to collect and organize an order for at least one medical equipment.

Yet another objective of the invention is to provide a simple method that provides real-time delivery of at least one medical equipment to at least one healthcare provider.

These and other advantages and features of the present invention are described with specificity so as to make the present invention understandable to one of ordinary skill in the art.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention, thus the drawings are generalized in form in the interest of clarity and conciseness.

FIG. 1 is a schematic diagram of an electronic data flow management system for streamlining a process of ordering, delivering and billing of at least one medical equipment; and

FIG. 2 is an operational flow chart of a method for ordering, delivering and billing of the at least one medical equipment utilizing the electronic data flow management system.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

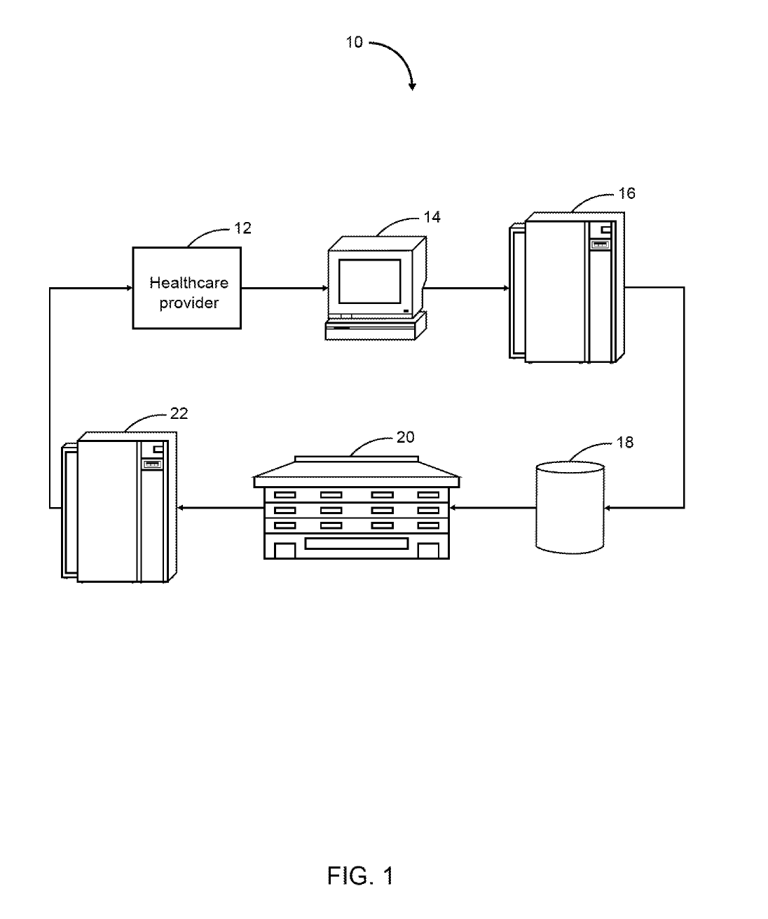

FIG. 1 is a schematic representation of an electronic data flow management system 10 for streamlining a process of ordering, delivering and billing of at least one medical equipment. The system 10 comprises at least one healthcare provider 12 having at least one workstation 14, a computer ordering server 16 associated with the at least one healthcare provider, an online database 18 and at least one durable medical equipment (DME) company 20 having a mainframe server 22. The at least one workstation 14 is adaptable to generate and sent an order for the at least one medical equipment. The computer ordering server 16 receives the order for the at least one medical equipment from the at least one workstation 14. The online database 18 receives and stores the order from the computer ordering server 16. The mainframe server 22 receives the order from the online database 18 and delivers the at least one medical equipment to at least one patient. The at least one DME company 20 utilizes a billing module to generate a bill for the at least one medical equipment.

The electronic data flow management system 10 provides a secure and cost effective way of procuring the at least one medical equipment to the at least one healthcare provider 12. The at least one medical equipment may be selected from a group consisting of wheel chairs, beds, crutches and oxygen tanks. The order may include information regarding the medical equipment, patients, physicians and insurance companies. The at least one healthcare provider 12 may be selected from a group consisting of hospitals, independent practice associations (IPA), doctors, nurses, nursing facilities, pharmacies, government agencies, and other hospital affiliated organizations. The DME company 20 provides real time delivery of the medical equipment. The DME company 20 sends the bill for the medical equipment to the healthcare provider 12. The website provides information about the order, the healthcare provider 12, the insurance company, patient and reauthorization request. The healthcare provider 12 sends the order in at least one mode. The at least one mode may be selected from a group consisting of an electronic mode and a fax mode. In the electronic mode, the healthcare provider sends the order electronically to the DME company and in the fax mode the order is sent as fax. The online database 18 is designed to map and/or parse the order for the medical equipment. The DME company 20 is approved by the healthcare provider 12. The system 10 implements electronic signatures for confirming the order for the medical equipment.

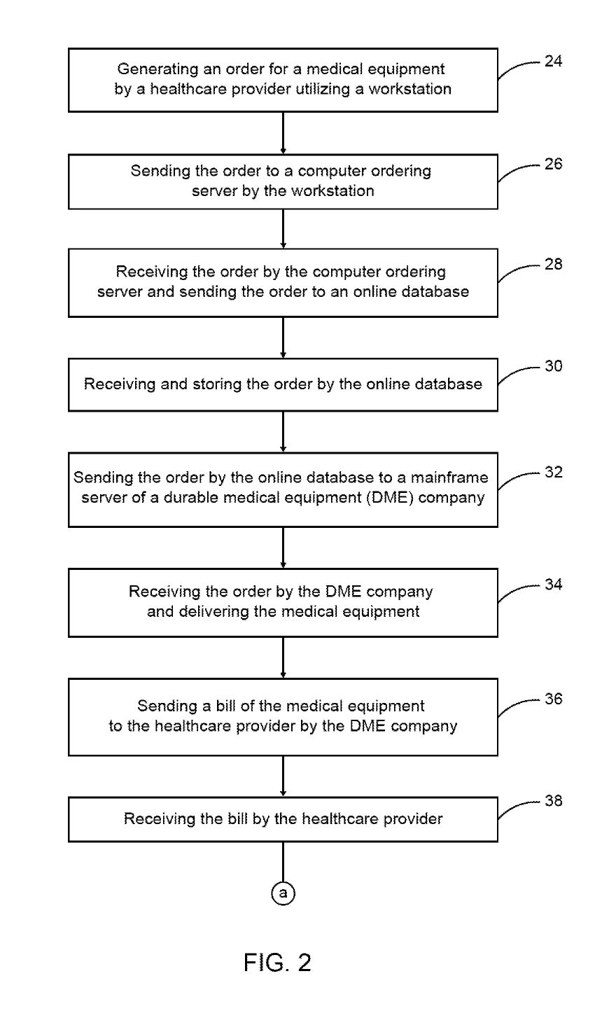

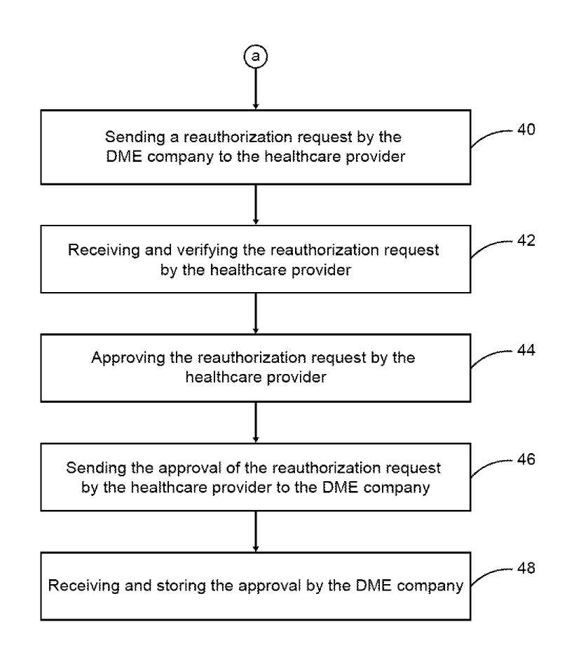

FIG. 2 shows an operational flow chart illustrating a method for ordering, delivering and billing of the medical equipment utilizing the electronic data flow management system 10. The healthcare provider utilizes the workstation to generate the order for the medical equipment as indicated at block 24. The workstation sends the order to the computer ordering server as indicated at block 26. The computer ordering server receives the order and sends to the online database as shown in block 28. As shown in block 30, the online database receives and stores the order and directs the order to the mainframe server as indicated at block 32. The DME company receives the order and delivers the medical equipment to the patient as indicated at block 34.

The DME company sends the bill of the medical equipment to the healthcare provider as indicated at block 36. The healthcare provider receives the bill as indicated at block 38. The DME company sends a reauthorization request to the healthcare provider as indicated at block 40. As indicated at block 42, the healthcare provider receives and verifies the reauthorization request. The healthcare provider approves the reauthorization request as shown in block 44 and sends the approval to the DME company as indicated at block 46. The DME company receives and stores the approval as indicated at block 48.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. An electronic data flow management system for streamlining a process of ordering, delivering and billing of at least one medical equipment, the system comprising:
at least one healthcare provider having at least one workstation, the at least one workstation being adaptable to generate and send an order for the at least one medical equipment;
a computer ordering server associated with the at least one healthcare provider to receive the order for the at least one medical equipment from the at least one workstation;
an online database to receive and store the order from the computer ordering server; and
at least one durable medical equipment (DME) company having a mainframe server, the mainframe server being able to receive the order from the online database, deliver the at least one medical equipment and generate a bill for the at least one medical equipment utilizing a website;
whereby the electronic data flow management system provides a secure and cost effective way of procuring the at least one medical equipment to the at least one healthcare provider.

2. The system of claim I wherein the at least one medical equipment may be selected from a group consisting of wheel chairs, beds, crutches and oxygen tanks.

3. The system of claim 1 wherein the order may include information regarding the at least one medical equipment, at least one patient, at least one physician and at least one insurance company.

4. The system of claim 1 wherein the at least one healthcare provider may be selected from a group consisting of hospitals, independent practice associations (IPA), doctors, nurses, nursing facilities, pharmacies, government agencies, and other hospital affiliated organizations.

5. The system of claim 1 wherein the at least one DME company provides real time delivery of the at least one medical equipment.

6. The system of claim I wherein the at least one DME company utilizes a billing module to send the bill for the at least one medical equipment to the at least one healthcare provider.

7. The system of claim 1 wherein the website provides information about the order, the at least one healthcare provider, the at least one insurance company, the at least one patient and at least one reauthorization request.

8. The system of claim 1 wherein the at least one healthcare provider sends the order in at least one mode.

9. The system of claim 8 wherein the at least one mode may be selected from a group consisting of an electronic mode and a fax mode.

10. The system of claim 1 wherein the online database is designed to map and/or parse the order for the at least one medical equipment.

11. The system of claim 1 wherein the at least one DME company is approved by the at least one healthcare provider.

12. A method for ordering, delivering and billing of at least one medical equipment utilizing an electronic data flow management system, the method comprising the steps of:
(a) generating an order for the at least one medical equipment by at least one healthcare provider having at least one workstation;
(b) sending the order to a computer ordering server associated with the at least one healthcare provider by the at least one workstation;
(c) receiving the order by the computer ordering server and directing the order to an online database;
(d) receiving and storing the order by the online database;
(e) sending the order to a mainframe server of at least one durable medical equipment (DME) company by the online database;
(f) receiving the order by the mainframe server and delivering the at least one medical equipment to at least one patient;
(g) sending a bill of the at least one medical equipment to the at least one healthcare provider by the at least one DME company;
(h) receiving the bill by the at least one healthcare provider;
(i) sending a reauthorization request by the at least one DME company to the at least one healthcare provider;
(j) receiving and verifying the reauthorization request by the at least one healthcare provider;
(k) approving the reauthorization request by the at least one healthcare provider;
(l) sending the approval of the reauthorization request by the at least one healthcare provider to the at least one DME company; and
(m) receiving and storing the approval by the at least one DME company.

13. The method of claim 12 wherein the order may include information regarding the at least one medical equipment, the at least one patient, at least one physician and at least one insurance company.

14. The method of claim 12 wherein the at least one medical equipment may be selected from a group consisting of wheel chairs, beds, crutches and oxygen tanks.

15. The method of claim 12 wherein the at least one healthcare provider may be selected from a group consisting of hospitals, independent practice associations (IPA), doctors, nurses, nursing facilities, pharmacies, government agencies and other hospital affiliated organizations.

16. The method of claim 12 wherein the at least one healthcare provider sends the order in at least one mode.

17. The system of claim 16 wherein the at least one mode may be selected from a group consisting of an electronic mode and a fax mode.

18. The method of claim 12 wherein the online database is designed to map and/or parse the order for the at least one medical equipment.

19. The method of claim 12 wherein the at least one DME company is approved by the at least one healthcare provider.

20. The method of claim 1 wherein the at least one DME company utilizes a billing module to send the bill of the at least one medical equipment to the at least one healthcare provider.
